(19) United States
(12) Patent Application Publication
(10) Pub. No.: US 2024/0165332 A1
(43) Pub. Date: May 23, 2024

(54) INJECTOR APPARATUS FACILITATING AUTOMATIC CANNULA WITHDRAWAL

(71) Applicant: Owen Mumford Limited, Woodstock, Oxfordshire (GB)

(72) Inventors: Toby COWE, Woodstock, Oxfordshire (GB); Anna COE, Woodstock, Oxfordshire (GB); Aylish MOORE, Woodstock, Oxfordshire (GB)

(21) Appl. No.: 18/547,961

(22) PCT Filed: Feb. 28, 2022
(86) PCT No.: PCT/EP2022/054940

§ 371 Date: Aug. 25, 2023

(30) Foreign Application Priority Data
Feb. 28, 2021 (GB) 2102845.1

(51) Int. Cl. A61M 5/20; A61M 5/24; A61M 5/315

(52) U.S. Cl. CPC A61M 5/2033; A61M 5/2422; A61M 5/31511; A61M 2005/2073

(57) ABSTRACT

An injector for use with a primary package. The injector comprises distal and proximal housing components in telescopic engagement, a rod configured for axial movement with the distal housing part, and a mechanism for releasably fixing the rod to a plunger to allow trained axial movement of the rod and the plunger during telescoping together of the distal and proximal housing components over a first distance whilst allowing relative axial movement of the rod and the plunger during telescoping together of the distal and proximal housing components over a second, subsequent distance. The mechanism comprises two or more flexible fingers on one of the rod and the plunger and cooperating features on the other of the rod and the plunger, and a locking component axially moveable during telescoping between a locking position in which it substantially surrounds the flexible fingers and a release position in which it is axially spaced from the locking fingers.

### TECHNICAL FIELD

The present invention relates to an injector apparatus facilitating automatic cannula withdrawal and in particular, though not necessarily, to such an injector apparatus that provides for manual injection of a dose from a cartridge or syringe.

### BACKGROUND

PCT/GB2019/050317 describes a so-called “primary package” comprising a cartridge for containing a medicament or “drug” in combination with a cannula component. This primary package 1 is illustrated in FIG. 1, where the cartridge is identified by reference numeral 2 and the cannula component by reference numeral 3. Considering each component in more detail, the cartridge comprises a generally cylindrical glass or plastics body 4 having a moveable bung 5 located at an end distal from the end of the body to be connected to the cannula component. The other end of the body 4 is shaped for connection to the cannula component 3 and is closed by a cap 6 comprising a sealing element 7 in the form of a disc shaped elastomeric septum. The cannula component 3 comprises a cannula 8 glued into a hub 9 which is in turn slidably located within a generally cylindrical body 9a. The cannula is enclosed within a safety cap or boot 10 which engages with the hub 9. The boot has an opening therein that is closed by an antibacterial barrier 11. The barrier 11 is permeable to air to prevent external pressure fluctuations from damaging the apparatus.

The cartridge is typically filled with a drug in a sterile environment, typically prior to sealing by the septum. Within this same sterile environment, the cap 6 with antimicrobial barrier is then secured to the body 4, after which the cannula component 3 is attached. In this state, the end of the cannula 8 is held off of the sealing element 7 so that there is no fluid communication between the two components 2,3.

WO2019155202 describes arrangements in which the primary package 1 is installed into a medicament delivery device such as a manually operated syringe or an automatic injector (“autoinjector”). After removal of the boot 10, a first stage of operation is the advancement of the primary package to a point where further progress of the cannula component is prevented whilst the cartridge continues to advance.

This causes the end of the cannula to penetrate the sealing element allowing the drug to enter the cannula. Injection can then proceed.

The known medicament delivery devices may not be ideal in situations where user dexterity is limited. This might happen for example in an emergency situation where a user is suffering an anaphylactic shock and wishes to deliver an emergency injection of epinephrine. In such situations, use of a “manual injector” is helpful. Such manual injectors allow for injection using only a single hand and with essentially a “stabbing” action where the device need only be grabbed in the user's fist. Manual injectors may also be more reliable and robust than aoutoinjectors.

A challenge that arises in designing a suitable manual injector is to ensure appropriate shielding of the cannula following injection so as to prevent cannula stick injuries and prevent contamination and cross-infection. Similar challenges arise when designing a manual injector suitable for use with a syringe.

WO9421316 describes an injection device for use with a syringe and which utilizes a main drive spring to facilitate cannula insertion and drug delivery. A return spring is configured to push the syringe and its cannula back into the main housing after the injection is complete.

### SUMMARY

According to a first aspect of the present invention there is provided an injector for use with a primary package or syringe comprising a cannula component and a body containing a medicament and a bung located within the body. The injector comprises a substantially cylindrical proximal component configured to accommodate said cannula component and at least a part of said body, a substantially cylindrical distal component for telescopic engagement with said proximal component, the distal component having a distal end wall and a member extending axially from said distal end wall into a central region of the distal component.

The injector is configured such that a force applied to telescope the proximal and distal components together causes a plunger to:
push the primary package or syringe components through the proximal component and thereby cause a cannula of the cannula component to project from the proximal component;
thereafter drive the bung through the body of the cartridge or syringe body to eject medication through the cannula component; and
thereafter disengage the plunger from the distal component to allow the entire primary package or syringe, together with the plunger, to be moved through the injector in a distal direction and thereby withdraw the cannula entirely into the proximal component.

One of said member and said plunger comprises two or more flexible fingers extending in a substantially axial direction and the other of said member and said plunger comprises engagement features for engaging with said fingers, the injector further comprising a locking component that is axially movable within said distal component between
a first position in which the locking component surrounds said fingers and said engagement features to prevent their relative radial movement and thereby secure the member to the plunger, and
a second position in which the locking component allows said relative movement and thereby at least partially disengages the member from the plunger.

The locking component is retained in said first position during steps a) and b), and at step c) being moved to said second position by axial abutment of the locking component with a feature provided on an interior surface of said proximal component.

Said proximal and distal components may comprise respective substantially cylindrical housings, one of the housings having a greater diameter than the other housing to facilitate said telescopic engagement. The engagement features may comprises a recess or recesses. The locking component may be an annular ring located substantially co-axially with the distal component.

The proximal component may be configured to restrain the flexible fingers during telescopic movement of the proximal and distal components together from said first to said second position.

The injector may comprise a compression spring located within said proximal component and being coupled to said primary package or syringe body in use such that the spring is compressed by said pushing of the primary package or syringe body through the proximal component, the injector being configured such that said disengagement of the plunger and said member allows the spring to expand and cause said movement of the entire primary package or syringe components, and the plunger, through the injector in a distal direction and thereby withdraw the cannula entirely into the proximal component. The spring may be a helical spring, optionally abutting an interior surface at a proximal end of the proximal component.

The injector may be assembled around a primary package or syringe. The injector may be a manual injector whereby said force applied to telescope the proximal and distal components together is a manually applied force.

The injector may be an autoinjector and comprising an energy storage mechanism and release mechanism for releasing energy from the storage mechanism to apply said force to telescope the proximal and distal components together.

The plunger may comprise a generally cylindrical body supporting said flexible fingers and said engagement features are provided by means of a profile formed around said member, said member optionally being an elongate member such as a rod.

The said member may be further coupled to said plunger by means of cooperating helical threads, the threads optionally having an adhesive applied between them, wherein following the at least partial disengagement, the plunger and the member remain coupled by the threads such that further trained axial movement of the member and the plunger continues until the plunger bottoms out, whereupon relative rotation occurs.

The injector may comprise a locking member for preventing relative axial movement of the plunger of the syringe and the syringe body during cannula insertion, the locking member being released upon completion of insertion. The locking member may comprise a surface for abutting said syringe body and two or more flexible legs deflectable into and out of engagement with said piston.

According to a second aspect of the present invention there is provided an injector for use with a primary package or syringe comprising a cannula component and a body containing a medicament and a bung located within the body, the injector having a plunger in the case of a primary package or making use of a plunger of the syringe in the case of a syringe. The injector comprises distal and proximal housing components in telescopic engagement, a rod configured for axial movement with the distal housing part, and a mechanism for releasably fixing the rod to the plunger to allow trained axial movement of the rod and the plunger during telescoping together of the distal and proximal housing components over a first distance whilst allowing relative axial movement of the rod and the plunger during telescoping together of the distal and proximal housing components over a second, subsequent distance. The mechanism comprises two or more flexible fingers on one of the rod and the plunger and cooperating features on the other of the rod and the plunger, and a locking component axially moveable during telescoping between a locking position in which it substantially surrounds the flexible fingers and a release position in which it is axially spaced from the locking fingers.

The injector may comprise a helical spring compressible during telescoping together of the distal and proximal housing components, the spring being configured to push the primary package and the plunger, or syringe, in a distal direction at a time after release of the rod from the plunger to withdraw the cannula component into the proximal housing component.

According to a third aspect of the present invention there is provided injector for use with a syringe comprising a cannula component, a body containing a medicament and a bung located within the body, and a plunger. The injector comprises distal and proximal housing components in telescopic engagement, and a rod configured for axial movement with the distal housing part. The injector further comprises:
a first mechanism for releasably fixing the rod to the plunger to allow trained axial movement of the rod and the plunger during telescoping together of the distal and proximal housing components over a first distance whilst allowing relative axial movement of the rod and the plunger during telescoping together of the distal and proximal housing components over a second, subsequent distance;
a second mechanism for maintaining engagement of the rod and the plunger following release by the first mechanism, the second mechanism comprising cooperating screw threads on the rod and the plunger to prevent or restrict relative axial movement of the rod and the plunger substantially until the plunger reaches an end of dose position, and thereafter allow the plunger and the rod to disengage by relative rotation.

The injector may comprise a helical spring compressible during telescoping together of the distal and proximal housing components, the spring being configured to push the primary package and the plunger, or syringe, in a distal direction at a time after the disengagement of the rod from the plunger, by said relative rotation, to withdraw the cannula component into the proximal housing component.

According to a further aspect of the present invention there is provided an injector for use with a primary package or syringe comprising a cannula component and a body containing a medicament and a bung located within the body, the injector having a plunger in the case of a primary package or making use of a plunger of the syringe in the case of a syringe, the injector comprising:
distal and proximal housing components in telescopic engagement;
a mechanism for releasably fixing the plunger relative to the primary package or syringe body to facilitate trained axial movement of the plunger and the primary package or the syringe body during a needle insertion phase and for releasing the plunger from the primary package or syringe body after needle insertion.

The said mechanism may comprise two or more flexible legs depending from a base member through which the plunger passes, and cooperating features on the plunger for engaging with the flexible legs.

According to a still further aspect of the present invention there is provided kit of parts for assembly into an injector according to any one of the preceding aspects, about a primary package or syringe.

According to a still further aspect of the present invention there is provided an injector for use with a primary package comprising a cannula component and a cartridge, the cartridge comprising a body containing a medicament and a bung located within the body. The injector comprises a proximal component configured to accommodate said cannula component and at least a part of said cartridge, a distal component for telescopic engagement with said proximal component, and a plunger releasably engaged within said distal component for engagement with said bung. The injector is configured such that a force applied to telescope the proximal and distal components together causes the plunger to act on the bung to push the primary package through the proximal component and thereby cause a cannula of the cannula component to project from the proximal component, thereafter drive the bung through the body of the cartridge to eject medication through the cannula component, and thereafter disengage the plunger from the distal component to allow the entire primary package and the plunger to be moved through the injector in a distal direction and thereby withdraw the cannula entirely into the proximal component.

The proximal and distal components may comprise respective substantially cylindrical housings, one of the housings having a greater diameter than the other housing to facilitate said telescopic engagement. The injector may further comprises one or more radially moveable fingers coupled to the cylindrical housing of the distal component and a feature or features provided in or on said plunger for engagement with the one or more radially moveable fingers to facilitate said releasable engagement of the plunger with the distal component. The one or more radially moveable fingers may be coupled to one or more respective flexible arms extending substantially axially through the cylindrical housing of the distal component from a distal end of the housing. The feature provided on the plunger may comprise a recess or recesses.

The injector may comprise a restraining component located within the distal component, the restraining component being movable in a distal direction within the distal component from an initial pre-injection position in which it restrains said fingers from radially outward movement to a post-injection position in which is does not restrain said fingers from radially outward movement. The restraining component may be an annular ring located co-axially with the cylindrical housing of the distal component. The device may be configured such that telescopic movement of the proximal and distal components together by a first predefined distance, corresponding to an end of injection distance, causes engagement of said restraining component with a feature or features of the proximal component, thereby moving the restraining component from said pre-injection position to said post-injection position. The cylindrical housing of the proximal component may be configured to further restrain the one or more flexible fingers during telescopic movement of the proximal and distal components together by a second predefined distance, following telescopic movement by said first predefined distance.

The injector may be configured such that further telescopic movement of the proximal and distal components together, following telescopic movement over said first or said second predefined distance, releases the one or more fingers to move radially outwards, causes said disengagement of the plunger and the distal component. Disengagement of the plunger and the distal component may allow the plunger to pass over or between the or each flexible arm.

The injector may comprise a compression spring, for example a helical compression spring, located within said proximal component and being coupled to said primary package in use such that the spring is compressed by said pushing of the primary package through the proximal component, the injector being configured such that said disengagement of the plunger and the distal component allows the spring to expand and cause said movement of the entire primary package and the plunger through the injector in a distal direction and thereby withdraw the cannula entirely into the proximal component. According to an aspect of the present invention, the injector is provided as a kit of parts for assembly about a primary package. Alternatively, the injector may be assembled around a primary package.

The injector may be a manual injector whereby said force applied to telescope the proximal and distal components together is a manual force.

The injector may be an autoinjector and comprising an energy storage mechanism and release mechanism for releasing energy from the storage mechanism to apply said force to telescope the proximal and distal components together.

According to a further aspect of the present invention there is provided an injector for use with a primary package comprising a cannula component and a cartridge, the cartridge comprising a body containing a medicament and a bung located within the body. The injector comprises a generally cylindrical proximal component defining an inner space for accommodating said cannula component and at least a part of said cartridge, a generally cylindrical distal component for telescopic engagement with said proximal component, and a plunger within an inner space defined by said distal component and having a proximal end for engagement with said bung. The injector further comprises one or more fingers fixed relative to said distal component, the finger(s) engaging a feature or features defined at a distal end of said plunger to provide releasable of engagement of the plunger within the distal component, and a locking mechanism disposed over said finger(s) to prevent disengagement from said features until the locking mechanism is engaged by a distal end of said proximal component and pushed in a distal direction within the distal component.

### BRIEF DESCRIPTION OF THE DRAWINGS

FIG. 1 is a cross-sectional view through a known primary package comprising a cartridge and a cannula component for assembly in a sterile environment;

FIG. 2 is a cross-sectional view through a proximal component of a manual injector for use with the primary package of FIG. 1;

FIG. 3 is a cross-sectional view through a distal component of a manual injector for use with the primary package of FIG. 1;

FIG. 4 is a cross-sectional view through a manual injector comprising the proximal component of FIG. 2 and the distal component of FIG. 3, where the injector comprises the (assembled) primary package of FIG. 1 and is in a pre-use state;

FIG. 5 is a cross-sectional view through the manual injector of FIG. 4 with an over-cap of the distal part removed;

FIGS. 6 to 10 are a cross-sectional views through the manual injector at various stages in an injection operation;

FIG. 11 is a perspective view of an assembled manual injector according to an alternative embodiment and for use with a primary package;

FIG. 12 is an exploded view of the manual injector if FIG. 11 including the primary package;

FIG. 13 shows a sequence of states of the manual injector of FIG. 11 in use;

FIG. 14 illustrates a plunger and rod of the injector of FIG. 11;

FIG. 15 is a perspective view of an assembled manual injector according to an alternative embodiment and for use with a syringe including a plunger;

FIG. 16 is an exploded view of the manual injector if FIG. 15 including the syringe; and

FIG. 17 shows a sequence of states of the manual injector of FIG. 15 in use.

### DETAILED DESCRIPTION

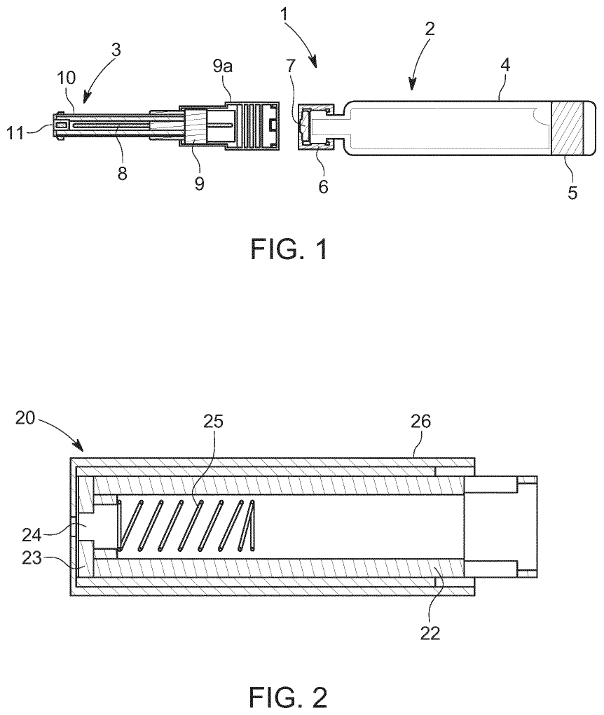

A known primary package 1 for containing a medicament or drug has been described above with reference to FIG. 1. A manual injector that facilitates an injection using such a known primary package, or indeed other primary packages with similar configuration, will now be described. For ease of understanding, reference will be made to proximal and distal ends of the injector and the package 1, where “proximal” refers to an end closest to the cannula tip and to the skin in use, whilst “distal” refers to an end furthest from the cannula tip and skin in use. It is noted that, in the context of the following discussion, the terms “needle” and “cannula” are used interchangeably and the use of one term encompasses the other, even though the precise technical meaning of each may differ.

FIG. 2 illustrates a proximal component 20 of the manual injector 21 and comprises a generally cylindrical housing 22 shaped to receive and accommodate the primary package 1. Whilst the housing is generally open at its distal end, it is provided at its proximal end with an end wall 23. The end wall is provided with a centrally located circular opening 24. A compression spring 25 is located within the housing 22 and, in an unassembled state, is in an uncompressed state as illustrated in FIG. 2. An overcap or “boot remover” 26, also of a generally cylindrical shape, is located concentrically over the housing 22 and is held in place by, for example, a suitable snap fit or interference fit connection. The proximal end of the boot remover 26 is closed so as to cover the circular opening 24. Whilst the spring 25 will likely, although not necessarily, be formed of a metal, other components of the proximal component will likely be formed using plastics materials and injection molding processes, although other materials and production processes may be used.

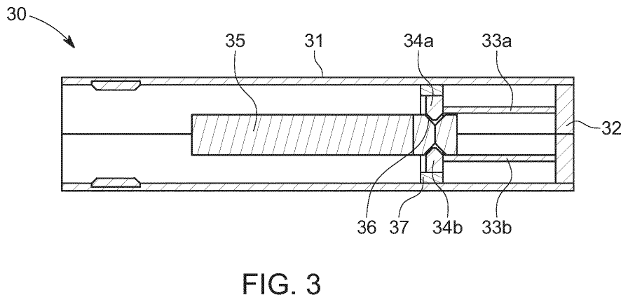

FIG. 3 illustrates a distal component 30 of the manual injector 21. This distal component comprises a generally cylindrical housing 31 that is closed at a distal end by a wall 32 and that is open at a proximal end. A pair of flexible arms 33a,b depend from the distal end wall 32 and extend axially, part-way through the cylindrical housing 31. [In some cases three or more arms may be provided.] The arms terminate with respective fingers 34a,b that extend both radially inward and outward to a small extent, i.e. the fingers have “T-like” shape. Located concentrically within the cylindrical housing 31 is a plunger 35 of generally cylindrical shape. The plunger is formed to provide a substantially V-shaped recess 36 extending circumferentially around a region adjacent a distal end of the plunger. The radially innermost ends of the fingers 34a,b are formed with a complimentary V-shape so that, during assembly, when the plunger is pushed into the housing 31 from the open end, the fingers 34a,b snap into and engage the recess 36. The plunger 35 is otherwise unconstrained within the housing 31. An annular ring 37 of outer diameter marginally less that the inner diameter of the housing 31 and of inner diameter marginally greater that the outer extent of the fingers 34a,b (when the fingers are engaged in the recess 36 of the plunger) surrounds the fingers and is prevented from moving in a proximal direction within the housing 31 by an appropriate feature or features of the housing. The ring 37 is also inhibited from moving within the housing in a distal direction by one or more features, although the resistance provided can be overcome to allow such movement as will be described below. Again, all of the parts of the distal component 30 will likely be formed using plastics materials and injection molding processes, although other materials and production processes may be used.

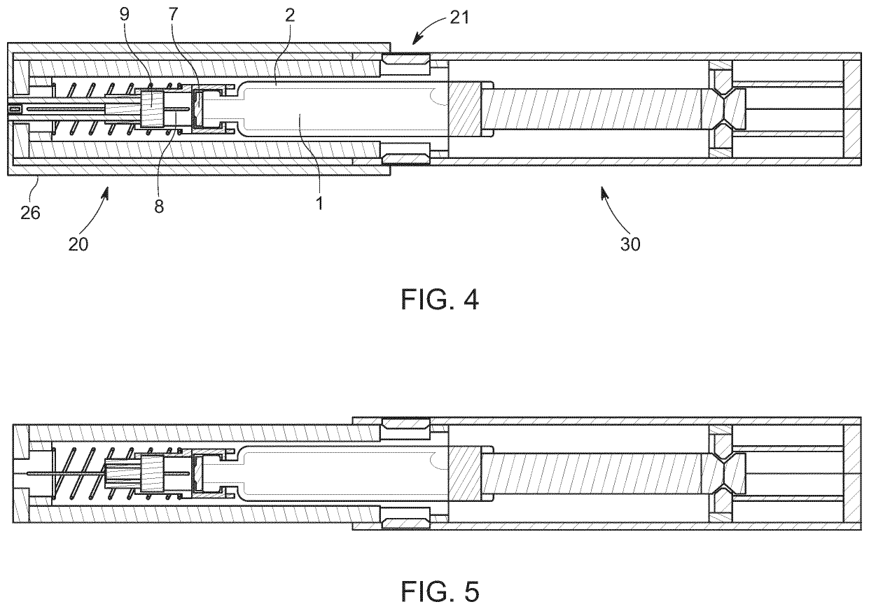

FIG. 4 illustrates the manual injector 21 assembled around a primary package 1. Assembly comprises firstly inserting the primary package into the open end of the proximal component 20. The primary package is pressed in so that features on the end of the boot 10 engage with complimentary features of the boot remover 26 in a known manner. The open end of the distal component 30 is positioned over and around the end of the proximal component such that a proximal end of the plunger 35 abuts the moveable bung 5 within the cartridge body 4. In this position, complimentary features of the distal and proximal component housings 22,31 are engaged to fix the two components together. These complimentary features are likely snap-fit features although other forms of engagement may be contemplated, e.g. screw threads. As will be apparent from FIG. 4, in this assembled, pre-use state, little or no force is applied between the cartridge 2 and the cannula component hub 9 (or cannula 8), such that the distal end of the cannula 8 remains spaced apart from the sealing element 7. The spring 25, which acts between the housing 31 and the cylindrical body 9a of the cannula component, is in a relaxed, generally uncompressed state. Features of the proximal and distal components, e.g. the same features fixing the components together, provide a resistance to further movement of the proximal component into the distal component which must be overcome to commence injection. Furthermore, any such movement is prevented by the boot remover 26 which is in blocking engagement with the housing 31 of the distal component. The configuration of the injector illustrated in FIG. 4 is typically that in which the injector is provided to an end user or healthcare professional. It will be appreciated that the design proposed here facilitates the use of low cost and environmentally friendly materials due to the absence of stored loads.

FIG. 5 illustrates a first step in the operation of the manual injector 21 and in which the boot remover 26 has been pulled off of the proximal component 20. This action also pulls the boot 10, which is captured by the boot remover, off of the cannula component 3, thereby exposing a proximal end of the cannula 8 within the proximal component 20. This breaks the sterile barrier. A latch between the hub 9 and the body 9a of the cannula component, or a simple “bump”, is provided to prevent the boot removal step from pulling the hub forward and potentially damaging the cannula or bringing it into contact with the sealing element 7.

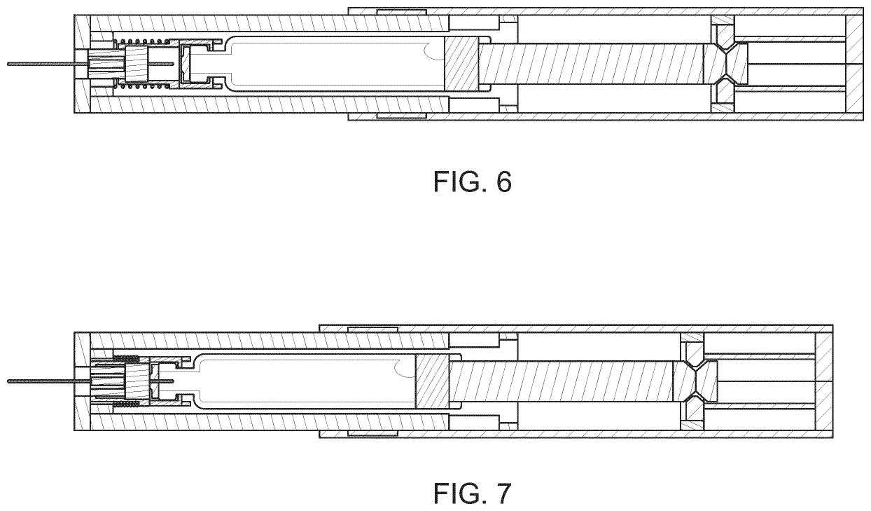

An injection can then be performed. This typically involves the user grasping the distal component 30 within his or her fist, and pressing the proximal end of the injector against the injection site. This is typically done in a stabbing motion such that cannula penetration and injection occurs in a quick and continuous manner. For the purpose of explanation, FIGS. 6 to 10 illustrate various stages in this injection process.

FIG. 6 illustrates a stage at which force applied between the proximal and distal components has caused the plunger 35, which is engaged by the fingers 34a,b, to advance into the proximal component, applying force to the bung 5 as it does so. The spring 25 initially offers little resistance to the primary package 1 such that the primary package is advanced through the proximal component. This results in the tip of the cannula 8 exiting through the opening 24 in the proximal component and penetrating the skin at the injection site. The tip of the cannula continues to advance until the needle hub 9 bottoms out on the end wall 23 of the housing 22. This is the position illustrated in FIG. 6.

As the user continues to apply force between the distal component and the skin, and thereby between the distal and proximal components, the hub 9, and with it the cannula 8, are pushed back through the cylindrical body 9a so that the distal tip of the cannula passes through the sealing element 7, bringing the flow channel of the cannula into fluid communication with the drug. This position is illustrated in FIG. 7. It is noted that the fingers 34a,b continue to be captured within the ring 37 which up to this point has not moved relative to the housing 31 of the distal component 30.

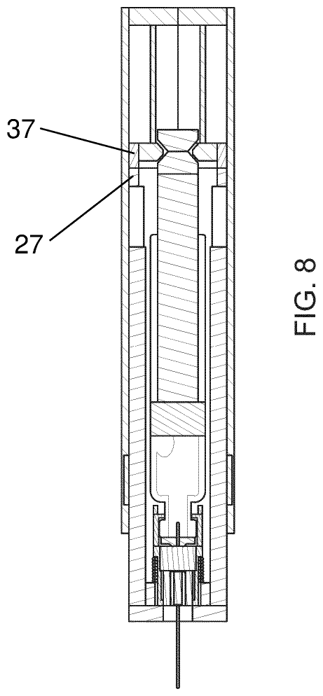

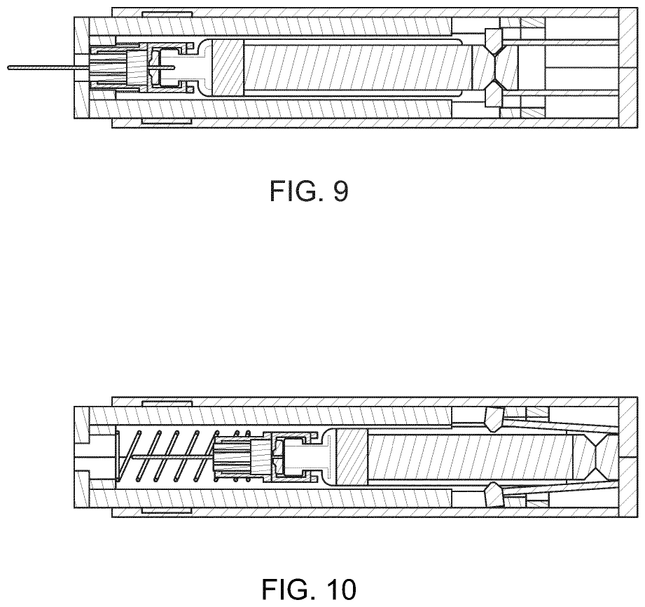

At this stage, further movement of the cannula and hub are prevented, as is any movement of the primary package 1 in a proximal direction. Further force applied by the user will now advance the plunger 35 and bung 5 through the cartridge body 4 of the primary package, thus delivering the drug through the cannula 8 and into the injection site. During this process the fingers 34a,b remain captured within the ring 37. Drug delivery is completed when the proximal component has advanced into the distal component by some predetermined distance. This is illustrated in FIG. 8. At this stage, the ring 37 is brought up against a distal end 27 of the proximal component whereupon continued applied force overcomes the resistance to distal movement of the ring within the housing 31 and moves the ring back within that housing. The fingers now pass through the distal end 27 where they continue to be restrained against any outward radial movement. However, on exiting the distal end 7, the fingers pass into the central space within the proximal component, whereupon any resistance to radially outward movement of the fingers 34a,b ceases. FIG. 9 illustrates a state of operation of the injector where the fingers are on the point of release from the recess 36 of the plunger 35. Continued relative movement of the proximal and distal components releases the fingers, substantially disengaging the plunger 35 and the housing 31 of the distal component 30.

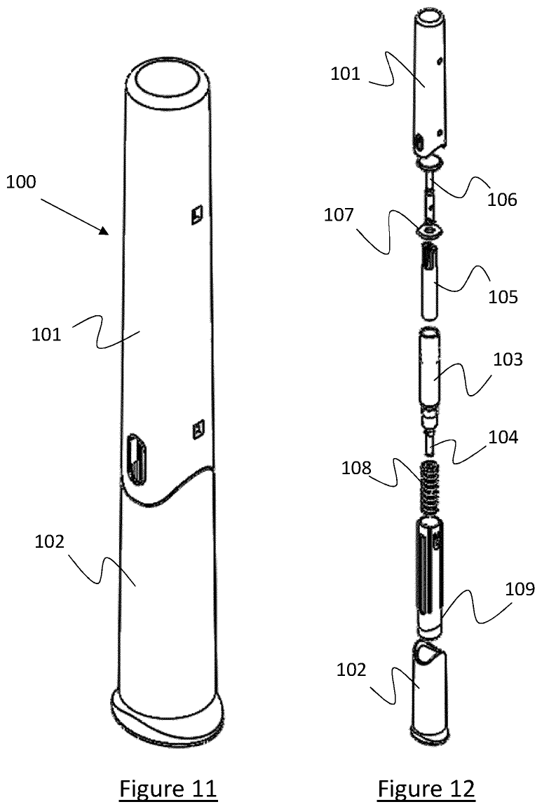

At this stage of operation, the spring 25, which is in substantially a fully compressed state, is free to expand, pushing the entire primary package 1 and the plunger 35 in a distal direction through the proximal component 20. The fingers are now driven out of the V-shaped recess by relative movement of the fingers and the plunger, with the supporting arms 33a,b splaying outwardly. The end of the plunger is accommodated within the space between the fingers 34a,b and the arms 33a,b, whilst the cannula is fully withdrawn into the proximal component where the tip of the cannula is no longer exposed. This state of operation of the injector is illustrated in FIG. 10. The possibility of stick injuries and contamination/cross-infection is eliminated or at least greatly reduced.

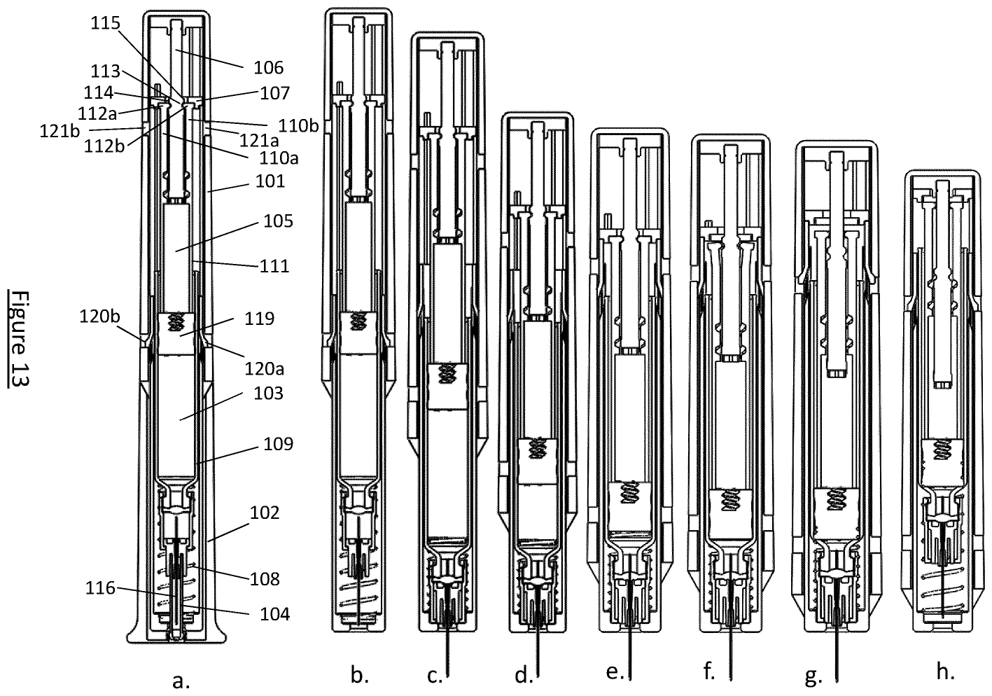

FIG. 11 illustrates an alternative embodiment of a manual injector 100 for use with a primary package such as that illustrated in FIG. 1. Visible in FIG. 11 are a distal housing part 101 and a boot remover 102. FIG. 12 is an exploded view of the manual injector, along the axis of the device. As well as the distal housing part 101 and the boot remover 102, visible in the exploded view are the primary package 103 including a boot 104 (covering a cannula which is not visible), a plunger 105, a member or rod 106, an annular ring 107, a helical metal spring 108, and a proximal housing part 109. The various components are configured to facilitate assembly of the device about the primary package by bringing the components together along the axis and such that they are secured together in a manner suitable for use, as will be apparent from the following explanation.

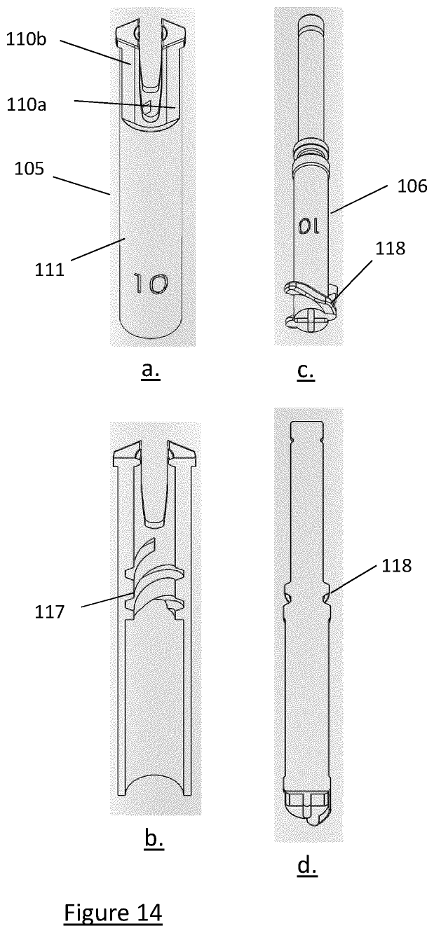

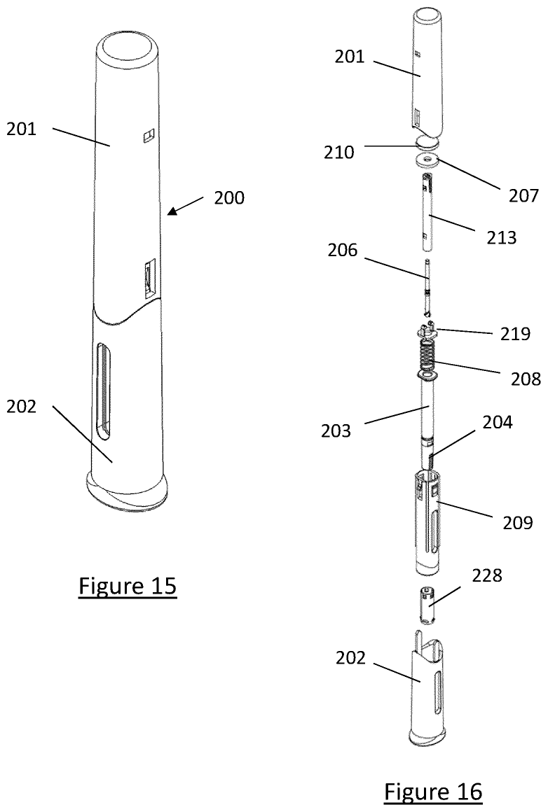

FIG. 13 illustrates the operation of the manual injector by way of a series of steps a. to h, as follows, where each illustration shows an axial cross-section of the device in the same plane:
a. A user receives the device in the fully assembled state including the primary package 103 and the boot remover 102. It will be apparent from this and other views that the rearmost end of the rod 106 (i.e. the distal end) abuts an inner end surface of the distal housing part 101 whilst a forwardmost end (i.e. proximal end) of the rod 106 is received by a pair of flexible legs 110a,b of the plunger 105, the flexible legs extending upwardly from a cylindrical body part 111 of the plunger. The flexible legs are provided with respective projections 112a,b extending radially inward to engage with an annular recess 113 formed around the outer surface of the rod 106. In the state shown in the illustration, the flexible legs are in an unstressed or relaxed state. The annular ring 107 is located around the rod 106 and is releasably retained at a required axial position on the rod by a circumferential ridge 114 formed on the rod and cooperating groove 115 formed around an inner surface of the ring 107. Alternative securing means may of course be used. In this state, the ring 107 also surrounds the projections 112a,b of the flexible legs, preventing the legs from flexing outwardly.
b. The user prepares the device for use by grasping the distal housing part 101 in one hand, gripping the boot remover 102 with the fingers of the other hand, and pulling the two parts apart thereby removing the boot remover and engaged boot 104 from the rest of the device and exposing the cannula 116 (but still shielded within the proximal housing part).
c. The user then presses the now exposed end of the proximal housing part 102 against the skin of the injection site, applying a large enough force to compress the helical spring 108 which is located between a shoulder of the main body of the primary package 103 and an end shoulder of the proximal housing part 109, and thereby telescoping the proximal and distal housing parts together. The distal housing part has a larger inner diameter than the proximal housing part such that the former slides over the latter during this telescoping movement. This action causes the cannula tip to extend outwardly, through an opening in the lower end of the proximal housing part 109, and penetrate the user's skin. It will be noted that the rod 106 is fixed axially relative to the plunger 105 by means of the annular ring 107 during this phase of operation, causing the rod, plunger and primary package (and the distal housing part) to move essentially as a single unit with respect to the proximal housing part until the cannula hub bottoms out at the end of the proximal housing part, i.e. with the spring substantially fully compressed. As described with respect to the previous embodiment, as the hub cannot move further, further movement of the body of the primary package causes the distal end of the cannula to penetrate a septum of the primary package bringing the interior channel of the cannula into fluid communication with the interior of the body and allowing medication to flow through the cannula into the user. It is noted that, during this stage of the operation, the proximal housing part is prevented from rotating relative to the distal housing part, e.g. by the location of anti-rotation projections of the proximal housing part within respective tracks extending coaxially along an inner surface of the distal housing part.
d. Continued force applied to the distal housing part 101 causes the continued telescoping of the housing parts together and medication delivery.
e. As an end of drug delivery position is approached, an uppermost end of the proximal housing part 109 makes contact with the annular ring 107.
f. Continued telescoping together of the housing parts causes the uppermost end of the proximal housing part to push the annular ring 107 up into the distal housing part after overcoming the relatively small resistance presented by the cooperating retaining features of the rod and the annular ring. As the flexible legs 110a,b are no longer prevented from flexing outwards by the annular ring, the rod 106 is now able to move down within the plunger, pushing the projections 112a,b outwardly as it progresses.
g. The flexible fingers and respective projections now flex inwardly behind the ridge formed on the rod, above the retaining recess. At this stage, in the absence of any mechanism to restrict relative axial movement of the rod 106 and the plunger 105, no further force could be applied to the plunger by the user as the rod would merely pass into the plunger. This is undesirable as, due to manufacturing tolerances, the annular ring may be freed from the flexible fingers prematurely and before the end of dose position has been reached (and the dose of medication fully delivered). Alternatively, the plunger may reach the end of the dose before the locking ring is released. To prevent either situation arising, the device is configured to ensure that the flexible fingers 111a,b are released before the end of dose is reached, taking into account the maximum possible manufacturing tolerances. To ensure that medication delivery can continue beyond this point, the plunger 105 comprises a short helical thread 117 on an inner surface thereof and which is engaged by a corresponding short thread 118 on an outer surface of the rod 107. The plunger is further illustrated in FIGS. 14a (perspective) and 14b (axial cross-section) whilst the rod 106 is further illustrated in FIGS. 14c (perspective) and 14d (axial cross-section). By carefully selecting the pitches of the threads, and if necessary introducing an adhesive to increase friction between the threads, it can be ensured that axial force exerted on the rod 106 will continue to be transferred to the plunger 105 and therefore the bung 119 even after release of the flexible fingers without necessarily resulting in relative rotation of the rod and the plunger. When the bung 119 and the plunger 105 bottom out, i.e. after (substantially) all medication has been ejected, continued force applied to the rod will result in its rotation relative through the plunger until the external thread 118 on the rod exits completely from the internal thread 117 on the plunger and into an interior space within the plunger. At this point, a pair of previously confined flexible legs 120a,b provided on an exterior surface of the proximal housing snap out into respective openings 121a,b provided in the distal housing part 101, preventing or restricting further relative axial movement of the proximal and distal housing parts.
h. As the threads on the plunger and the rod are now no longer engaged, these two parts are free to move axially relative to one another. This movement is driven by the compressed spring 108 which is now free to expand, pushing the plunger 105 rearwards over the rod 106 and back into the distal housing part 101. This results in the cannula 116 being withdrawn fully into the proximal housing part 109 so that no part of the cannula is exposed and preventing risk of a cannula stick injury to the user.

FIG. 15 is a perspective view of a still further embodiment of a manual injection device 200 and which is suitable for use with a syringe rather than a primary package. As such, the plunger forms an integral part of the syringe rather than the manual injection device itself. However, the skilled person will appreciate that the features and operation of this injection device are similar to those of the embodiment described above with respect to FIGS. 11 to 14 and the following description should be understood in light of the previously described embodiment.

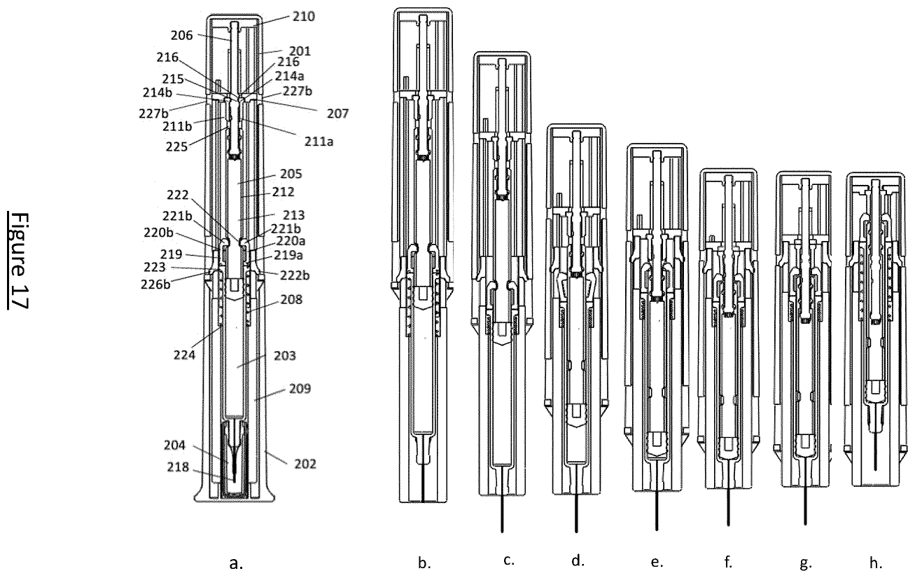

Visible in FIG. 15 are a distal housing part 201 and a boot remover 202. FIG. 16 is an exploded view of the manual injector, along the axis of the device 200. As well as the distal housing part 201 and the boot remover 202, visible in the exploded view are the syringe 203 including a boot 204 (covering a cannula which is not visible) and a plunger 213, a member or rod 206, an annular ring 207, a helical metal spring 208, and a proximal housing part 209 and a rod end cap 210 Also shown in the Figure is thin cylindrical metal pressing 228 which grips onto the cap end 202 with outwardly facing teeth, and onto the boot with inwardly facing teeth. This provides a reliable method to remove the boot. The various components are configured to facilitate assembly of the device 200 about the syringe 203 by bringing the components together along the axis and such that they are secured together in a manner suitable for use, as will be apparent from the following explanation.

FIG. 17 illustrates the operation of the manual injector by way of a series of steps a. to h, as follows, where each illustration shows an axial cross-section of the device:
a. A user receives the device in the fully assembled state including the syringe 203 and the boot remover 202. It will be apparent from this and other views that the rearmost end of the rod 206 is fixed to the rod end cap 210 which abuts an inner end surface of the distal housing part 201 whilst a forwardmost end of the rod 206 is received by a pair of flexible legs 211a,b of a plunger 205 of the syringe, the flexible legs extending upwardly from a cylindrical body part 212 of the plunger 213. The flexible legs are provided with respective projections 214a,b extending radially inward to engage with an annular recess 215 formed around the outer surface of the rod 206. In the state shown in the illustration, the flexible legs are in an unstressed or relaxed state. The annular ring 207 is located around the rod 206 and is releasably retained at a required axial position on the rod by a circumferential ridge 216 formed on the rod and cooperating groove 217 formed around an inner surface of the ring 207. In this state, the ring 207 also surrounds the projections 214a,b of the flexible legs, preventing the legs from flexing outwardly.
b. The user prepares the device for use by grasping the proximal housing part 201 in one hand, gripping the boot remover 202 with the fingers of the other hand, and pulling the two parts apart thereby removing the boot remover 202 and engaged boot 204 from the rest of the device and exposing the cannula 218 (still shielded within the proximal housing part).
c. In contrast to a primary package such as that shown in FIG. 1, the syringe will typically be provided with the distal end of the cannula already in fluid communication with the interior of the syringe body. Therefore, in order to prevent fluid from being expelled through the cannula during the cannula insertion phase, a mechanism is provided to prevent axial relative movement of the syringe body and plunger until this phase is completed. This mechanism comprises a locking member 219 having a generally rectangular base 219a defining a central opening therethrough, with a pair of upstanding legs 220a,b extending from the base. In the pre-use state, projections 221a,b are engaged within a recess 222 formed around the outer circumference of the plunger which otherwise passes through the central opening in the locking member. The base of the locking member 219 abuts an upper surface of the flange (finger grip) 223 surrounding the end of the syringe body. The legs 220a,b are prevented from being deflected outwardly by the surrounding regions of the proximal housing part 209 during initial forward movement of the distal housing part 201 relative to the proximal housing part 209. The compression spring 208 is located between the lower surface of the flange 223 and a shoulder 224 formed around an inner surface of the proximal housing part 209 such that the spring 208 is compressed during cannula insertion.
d. Following cannula insertion, the legs 220a,b move out of a confined region within the proximal housing part 209 into a region where they are not so confined. The legs are thus allowed to splay outwardly, releasing the locking member 219, and therefore the syringe body, from the plunger 213. Further force applied by the user to the distal housing part 201 therefore causes the plunger 213, driven by the rod 206, to move forwards within the syringe body, thereby ejecting medication through the cannula 218. It will be appreciated that the rod and plunger are secured to move axially together by means of the flexible legs and annular ring mechanism described above with respect to FIGS. 11 to 13. It is noted that, when splayed out, the projections 221a,b of the respective legs now engage respective recesses provided in the proximal housing part 209. The syringe body is therefore prevented from moving rearwards into the proximal housing part. This is important as it prevents withdrawal of the cannula (from the skin) should the user significantly reduce the applied force midway through the insertion step.
e. At the end of delivery, the projections 221a,b of the respective legs snap into a circumferential groove 225 extending around a distal part of the plunger 213, simultaneously disengaging the locking member, and therefore the syringe body, from the proximal housing part.
f. As described in respect of the embodiment of FIGS. 11 to 14, the locking ring 107 is now pushed rearwards with respect to the rod 206, releasing the engagement between the rod and the plunger. Continued force applied to the distal housing part 201 causes the rod 206 to begin rotating through the plunger 213 by means of the cooperating helical threads.
g. As the cooperating threads disengage, the distal and proximal housing parts arrive at a relative position where a pair of flexible lugs 226a,b extending from an exterior surface of the proximal housing part snap outward to engage respective openings 227a,b provided in the distal housing part, thereby securing the distal and proximal housing parts together.
h. The rod 206 is now fully disengaged from the plunger 213. This allows the spring 208 to expand, pushing the entire syringe, including the plunger 213, rearwards into the proximal housing part, fully shielding the cannula 218.

The mechanism described with respect to the accompanying figures provides a very simple and elegant solution to the problem of withdrawing and shielding a cannula following injection and which involves releasing the injector within the device once drug delivery is complete. Whilst it is well suited for use with a manual injector, the mechanism might also be usefully incorporated into an autoinjector which incorporates some force delivery mechanism, such as a spring, to push the plunger through the device.

It will be appreciated by the skilled person that various modifications may be made to the above described embodiments without departing from the scope of the present invention.

1-21. (canceled)

22. An injector for use with a primary package comprising a cannula component and a cartridge, the cartridge comprising a body containing a medicament and a bung located within the body, the injector comprising:
a proximal component configured to accommodate said cannula component and at least a part of said cartridge;
a distal component for telescopic engagement with said proximal component; and
a plunger releasably engaged within said distal component for engagement with said bung,
the injector being configured such that a force applied to telescope the proximal and distal components together causes the plunger to act on the bung to:
push the primary package through the proximal component and thereby cause a cannula of the cannula component to project from the proximal component;
thereafter drive the bung through the body of the cartridge to eject medication through the cannula component; and
thereafter disengage the plunger from the distal component to allow the entire primary package and the plunger to be moved through the injector in a distal direction and thereby withdraw the cannula entirely into the proximal component,
wherein said proximal and distal components comprise respective substantially cylindrical housings, and the injector further comprises:
said one or more radially moveable fingers being coupled to one or more respective flexible arms extending substantially axially through the cylindrical housing of the distal component from a distal end of the housing;
a feature or features provided in or on said plunger for engagement with the one or more radially moveable fingers to facilitate said releasable engagement of the plunger with the distal component; and
a restraining component located within the distal component, the restraining component being movable in a distal direction within the distal component from an initial pre-injection position in which it restrains said fingers from radially outward movement to a post-injection position in which is does not restrain said fingers from radially outward movement.

23. An injector according to claim 22, wherein said one of the housings has a greater diameter than the other housing to facilitate said telescopic engagement.

24. An injector according to claim 22, wherein said feature provided on the plunger comprises a recess or recesses.

25. An injector according to claim 22, said restraining component being an annular ring located co-axially with the cylindrical housing of the distal component.

26. An injector according to claim 22, said device being configured such that telescopic movement of the proximal and distal components together by a first predefined distance, corresponding to an end of injection distance, causes engagement of said restraining component with a feature or features of the proximal component, thereby moving the restraining component from said pre-injection position to said post-injection position.

27. An injector according to claim 26, said cylindrical housing of the proximal component being configured to further restrain the one or more flexible fingers during telescopic movement of the proximal and distal components together by a second predefined distance, following telescopic movement by said first predefined distance.

28. An injector according to claim 22, whereupon further telescopic movement of the proximal and distal components together, following telescopic movement over said first or said second predefined distance, releases the one or more fingers to move radially outwards, causing said disengagement of the plunger and the distal component.

29. An injector according to claim 22, wherein disengagement of the plunger and the distal component allows the plunger to pass over or between the or each flexible arm.

30. An injector according to claim 22 and comprising a compression spring located within said proximal component and being coupled to said primary package in use such that the spring is compressed by said pushing of the primary package through the proximal component, the injector being configured such that said disengagement of the plunger and the distal component allows the spring to expand and cause said movement of the entire primary package and the piston through the injector in a distal direction and thereby withdraw the cannula entirely into the proximal component.

31. An injector according to claim 29, where said spring is a helical spring.

32. An injector according to claim 22, the injector being assembled around a primary package.

33. An injector according to claim 22, the injector being a manual injector whereby said force applied to telescope the proximal and distal components together is a manual force.

34. An injector according to claim 22, the injector being an autoinjector and comprising an energy storage mechanism and release mechanism for releasing energy from the storage mechanism to apply said force to telescope the proximal and distal components together.
